(19) United States
(12) Patent Application Publication
(10) Pub. No.: US 2006/0210638 A1
(43) Pub. Date: Sep. 21, 2006

(54) Injectable compositions of nanoparticulate immunosuppressive compounds

(75) Inventors: Gary G. Liversidge, West Chester, PA (US); Scott Jenkins, Downingtown, PA (US)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International Limited

(21) Appl. No.: 11/376,554

(22) Filed: Mar. 16, 2006

Related U.S. Application Data
(60) Provisional application No. 60/662,692, filed on Mar. 17, 2005.

(51) Int. Cl. A61K 9/14; A61K 31/4745

(52) U.S. Cl. 424/489; 514/291; 977/906

(57) ABSTRACT

The invention is directed to an injectable nanoparticulate immunosuppressant composition for the formation of a subcutaneous or intramuscular depot. The invention is also directed to an injectable composition of nanoparticulate tacrolimus and/or sirolimus which eliminates the need to use polyoxyl 60 hydrogenated castor oil (HCO-60) and/or polysorbate 80 as a solubilizer. This invention further discloses a method of making an injectable nanoparticulate tacrolimus and/or sirolimus composition and is also directed to methods of treatment using the injectable nanoparticulate formulations comprising tacrolimus, sirolimus, or combination thereof for a subcutaneous or intramuscular depot for the prophylaxis of organ rejection and for the treatment of psoriasis or other immune diseases

### FIELD OF THE INVENTION

The invention is directed to injectable nanoparticulate compositions comprising at least one immunosuppressive compound. In an exemplary embodiment, the invention describes an injectable composition of a nanoparticulate immunosuppressive compound, such as tacrolimus, sirolimus, or a combination thereof.

### BACKGROUND OF THE INVENTION

A. Background Regarding Immunosuppressive Compounds

Examples of immunosuppressive compounds include, but are not limited to, tacrolimus and sirolimus.

1. Background Regarding Tacrolimus

Tacrolimus, or FK-506, is a macrolide immunosuppressant which is reputed to be 100 times more effective than cyclosporine. It is produced by fermentation of Streptomyces tsukubaensis, a monotypic species of Streptomyces. U.S. Pat. No. 4,894,366 and EPO Publication No. 0184162 describe tacrolimus and are incorporated by reference in their entirety.

Tacrolimus is sold under the trade name PROGRAF® (available from Fujisawa USA, Inc.) and suppresses some humoral immunity and, to a greater extent, cell-mediated reactions such as allograft rejection, delayed-type hypersensitivity, collagen-induced arthritis, experimental allergic encephalomyelitis, and graft versus host disease. Tacrolimus prolongs survival of a host and transplanted graft in animal transplant models of liver, kidney, heart, bone marrow, small bowel and pancreas, lung and trachea, skin, cornea, and limb.

Experimental evidence suggests that tacrolimus binds to an intracellular protein, FKBP-12. A complex oftacrolimus-FKBP-12, calcium, calmodulin, and calcineurin is then formed, and the phosphatase activity of calcineurin inhibited. This effect may prevent dephosphorylation and translocation of nuclear factor of activated T-cells (NF-AT), a nuclear component thought to initiate gene transcription for the formation of lymphokines (such as interleukin-2, gamma interferon). The net result is the inhibition of T-lymphocyte activation (i.e., immunosuppression).

Tacrolimus has an empirical formula of C₄₄H₆₉NO₁₂ .H₂O and a formula weight of 822.05. Tacrolimus appears as white crystals or crystalline powder and is practically insoluble in water, freely soluble in ethanol, and very soluble in methanol and chloroform. Tacrolimus has the following chemical structure: 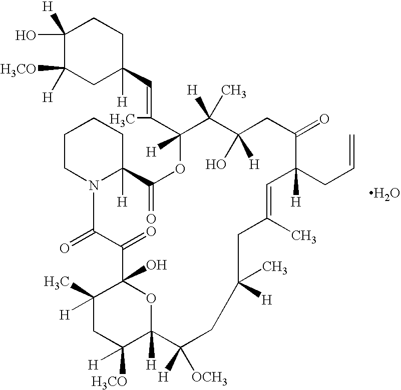
(See, The Merck Index, Twelfth Edition, 9200 (Merck & Co., Inc., Rahway, NJ, 1996).

Absorption of tacrolimus from the gastrointestinal tract after oral administration is incomplete and variable. The absolute bioavailability of tacrolimus is 17±10% in adult kidney transplant patients (N=26), 22±6% in adult liver transplant patients (N=17), and 18±5% in healthy volunteers (N=16).

A single dose study conducted in 32 healthy volunteers established the bioequivalence of the 1 mg and 5 mg capsules. Another single dose study in 32 healthy volunteers established the bioequivalence of the 0.5 mg and 1 mg capsules. Tacrolimus maximum blood concentrations (Cₘₐₓ) and area under the curve (AUC) appeared to increase in a dose-proportional fashion in 18 fasted healthy volunteers receiving a single oral dose of 3 mg, 7 mg, and 10 mg.

In 18 kidney transplant patients, tacrolimus trough concentrations from 3 to 30 ng/mL measured at 10-12 hours post-dose (Cₘᵢₙ) correlated well with the AUC (correlation coefficient 0.93). In 24 liver transplant patients over a concentration range of 10 to 60 ng/mL, the correlation coefficient was 0.94.

With respect to food effects, the rate and extent of tacrolimus absorption were greatest under fasted conditions. The presence and composition of food decreased both the rate and extent of tacrolimus absorption when administered to 15 healthy volunteers. The effect was most pronounced with a high-fat meal (848 kcal, 46% fat): mean AUC and Cₘₐₓ were decreased 37% and 77%, respectively; and Tₘₐₓ was lengthened 5-fold. A high-carbohydrate meal (668 kcal, 85% carbohydrate) decreased mean AUC and mean Cₘₐₓ by 28% and 65%, respectively.

In healthy volunteers (N=16), the time of the meal also affected tacrolimus bioavailability. When given immediately following the meal, mean Cₘₐₓ was reduced 71%, and mean AUC was reduced 39%, relative to the fasted condition. When administered 1.5 hours following the meal, mean Cₘₐₓ was reduced 63%, and mean AUC was reduced 39%, relative to the fasted condition.

In 11 liver transplant patients, tacrolimus administered 15 minutes after a high fat (400 kcal, 34% fat) breakfast, resulted in decreased AUC (27±18%) and Cₘₐₓ (50±19%), as compared to a fasted state.

Plasma protein binding of tacrolimus is approximately 99% and is independent of concentration over a range of 5-50 ng/mL. Tacrolimus is bound mainly to albumin and alpha-1-acid glycoprotein, and has a high level of association with erythrocytes. The distribution of tacrolimus between whole blood and plasma depends on several factors, such as hematocrit, temperature at the time of plasma separation, drug concentration, and plasma protein concentration. In a U.S. study, the ratio of whole blood concentration to plasma concentration averaged 35 (range 12 to 67).

Tacrolimus is extensively metabolized by the mixed-function oxidase system, primarily the cytochrome P-450 system (CYP3A). A metabolic pathway leading to the formation of 8 possible metabolites has been proposed. Demethylation and hydroxylation were identified as the primary mechanisms of biotransformation in vitro. The major metabolite identified in incubations with human liver microsomes is 13-demethyl tacrolimus. In in vitro studies, a 31-demethyl metabolite has been reported to have the same activity as tacrolimus.

The mean clearance following IV administration of tacrolimus is 0.040, 0.083, and 0.053 L/hr/kg in healthy volunteers, adult kidney transplant patients, and adult liver transplant patients, respectively. In man, less than 1% of the dose administered is excreted unchanged in urine.

In a mass balance study of IV administered radio-labeled tacrolimus to 6 healthy volunteers, the mean recovery of radiolabel was 77.8±12.7%. Fecal elimination accounted for 92.4±1.0% and the elimination half-life based on radioactivity was 48.1±15.9 hours whereas it was 43.5±11.6 hours based on tacrolimus concentrations. The mean clearance of radiolabel was 0.029±0.015 L/hr/kg and clearance of tacrolimus was 0.029±0.009 L/hr/kg. When administered orally, the mean recovery of the radiolabel was 94.9±30.7%. Fecal elimination accounted for 92.6±30.7%, urinary elimination accounted for 2.3±1.1% and the elimination half-life based on radioactivity was 31.9±10.5 hours, whereas it was 48.4±12.3 hours based on tacrolimus concentrations. The mean clearance of radiolabel was 0.226±0.116 L/hr/kg and clearance of tacrolimus 0.172±0.088 L/hr/kg.

In patients unable to take oral PROGRAF® capsules, therapy may be initiated with PROGRAF® injection. When considering the uses of PROGRAF® injection, it should be noted that anaphylactic reactions have occurred with tacrolimus injectables containing castor oil derivatives, such as CREMAPHOR®. Therefore, PROGRAF® injection is contraindicated in patients with a hypersensitivity to HCO-60 (polyoxyl 60 hydrogenated castor oil). The initial dose of PROGRAF® should be administered no sooner than 6 hours after transplantation. The recommended starting dose of PROGRAF® injection is 0.03-0.05 mg/kg/day as a continuous IV infusion. Adult patients should receive doses at the lower end of the dosing range. Concomitant adrenal corticosteroid therapy is recommended early post-transplantation Continuous intravenous (IV) infusion of PROGRAF® injection should be continued only until the patient can tolerate oral administration of PROGRAF® capsules.

PROGRAF® injection must be diluted with 0.9% Sodium Chloride Injection or 5% Dextrose Injection to a concentration between 0.004 mg/mL and 0.02 mg/mL prior to use. Diluted infusion solution should be stored in glass or polyethylene containers and should be discarded after 24 hours. The diluted infusion solution should not be stored in a PVC container due to decreased stability and the potential for extraction of phthalates. In situations where more dilute solutions are utilized (e.g., pediatric dosing, etc.), PVC-free tubing should likewise be used to minimize the potential for significant drug adsorption onto the tubing. Parenteral drug products should be inspected visually for particulate matter and discoloration prior to administration, whenever solution and container permit. Due to the chemical instability of PROGRAF® in alkaline media, PROGRAF® injection should not be mixed or co-infused with solutions of pH 9 or greater (e.g., ganciclovir or acyclovir).

If IV therapy is necessary, conversion from IV to oral tacrolimus is recommended as soon as oral therapy can be tolerated. In a patient receiving an IV infusion, the first dose of oral therapy should be given 8-12 hours after discontinuing the IV infusion. The recommended starting oral dose of tacrolimus capsules is 0.10-0.15 mg/kg/day administered in two divided daily doses every 12 hours. Co-administered grapefruit juice has been reported to increase tacrolimus blood trough concentrations in liver transplant patients. Dosing should be titrated based on clinical assessments of rejection and tolerability.

2. Background Regarding Sirolimus

Sirolimus is an immunosuppressive, macrolytic lactone produced by Streptomyces hygroscopicus. The chemical name of sirolimus (also known as rapamycin) is (3S,6R,7E,9R,10R,12R,14S,15E,17E,19E,21S,23S,26R,27R ,34aS)-9,10,12,13,14,21,22,23,24,25,26,27,32,33,34,34a-hexadecahydro-9,27-dihydroxy-3-[(1 R )-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c] [1,4] oxaazacyclohentriacontine-1,5,11,28,29(4H,6H,31H)-pentone. Its molecular formula is C₅₁H₇₉NO₁₃ and its molecular weight is 914.2. The structural formula of sirolimus is shown below.

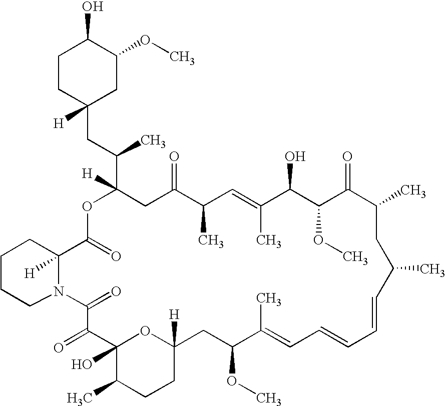

Sirolimus is a white to off-white powder and is insoluble in water, but freely soluble in benzyl alcohol, chloroform, acetone, and acetonitrile. Sirolimus is currently available as an oral dosage form sold under the tradename Rapamune® by Wyeth-Ayerst Inc. (Madison, N.J.). Rapamune® is available for administration as an oral solution containing 1 mg/mL sirolimus. Rapamune is also available as a white, triangular-shaped tablet containing 1-mg sirolimus, and as a yellow to beige triangular-shaped tablet containing 2-mg sirolimus.

The inactive ingredients in Rapamune® Oral Solution are Phosal 50 PG® (phosphatidylcholine, propylene glycol, mono- and di-glycerides, ethanol, soy fatty acids, and ascorbyl palmitate) and polysorbate 80. Rapamune Oral Solution contains 1.5%-2.5% ethanol. The inactive ingredients in Rapamune® Tablets include sucrose, lactose, polyethylene glycol 8000, calcium sulfate, microcrystalline cellulose, pharmaceutical glaze, talc, titanium dioxide, magnesium stearate, povidone, poloxamer 188, polyethylene glycol 20,000, glyceryl monooleate, carnauba wax, and other ingredients. The 2 mg dosage strength also contains iron oxide yellow 10 and iron oxide brown 70.

Sirolimus inhibits T-lymphocyte activation and proliferation that occurs in response to antigenic and cytokine (Interleukin [IL]-2, IL-4, and IL-15) stimulation by a mechanism that is distinct from that of other immunosuppressants. Sirolimus also inhibits antibody production. In cells, sirolimus binds to the immunophilin, FK Binding Protein-12 (FKBP-12), to generate an immunosuppressive complex. The sirolimus:FKBP-12 complex has no effect on calcineurin activity. This complex binds to and inhibits the activation of the mammalian target of sirolimus (mTOR), a key regulatory kinase. This inhibition suppresses cytokine-driven T-cell proliferation, inhibiting the progression from the G₁ to the S phase of the cell cycle.

Studies in experimental models show that sirolimus prolongs allograft (kidney, heart, skin, islet, small bowel, pancreatico-duodenal, and bone marrow) survival in mice, rats, pigs, and/or primates. Sirolimus reverses acute rejection of heart and kidney allografts in rats and prolongs the graft survival in presensitized rats. In some studies, the immunosuppressive effect of sirolimus lasts up to 6 months after discontinuation of therapy. This toleration effect is alloantigen specific.

In rodent models of autoimmune disease, sirolimus suppresses immune-mediated events associated with systemic lupus erythematosus, collagen-induced arthritis, autoimmune type I diabetes, autoimmune myocarditis, experimental allergic encephalomyelitis, graft-versus-host disease, and autoimmune uveoretinitis.

Sirolimus pharmacokinetic activity has been determined following oral administration in healthy subjects, pediatric dialysis patients, hepatically-impaired patients, and renal transplant patients. Sirolimus is rapidly absorbed following administration of Rapamune® Oral Solution, with a mean time-to-peak concentration (Tₘₐₓ) of approximately 1 hour after a single dose in healthy subjects and approximately 2 hours after multiple oral doses in renal transplant recipients. The systemic availability of sirolimus was estimated to be approximately 14% after the administration of Rapamune® Oral Solution. The mean bioavailability of sirolimus after administration of the tablet is about 27% higher relative to the oral solution. Sirolimus oral tablets are not bioequivalent to the oral solution; however, clinical equivalence has been demonstrated at the 2-mg dose level.). Sirolimus concentrations, following the administration of Rapamune® Oral Solution to stable renal transplant patients, are dose proportional between 3 and 12 mg/m².

B. Background Regarding Nanoparticulate Active Agent Compositions

Nanoparticulate active agent compositions, first described in U.S. Pat. No. 5,145,684 (“the '684 patent”), comprise particles of a poorly soluble therapeutic or diagnostic agent having adsorbed onto or associated with the surface thereof a non-crosslinked surface stabilizer. The '684 patent also describes methods of making such nanoparticulate active agent compositions but does not describe compositions comprising tacrolimus in nanoparticulate form. Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using lodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−) Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” U.S. Pat. No. 6,432,381 for “Methods for Targeting Drug Delivery to the Upper and/or Lower Gastrointestinal Tract;” U.S. Pat. No. 6,582,285 for “Apparatus for Sanitary Wet Milling;” and U.S. Pat. No. 6,592,903 for “Nanoparticulate Dispersions Comprising a Synergistic Combination of a Polymeric Surface Stabilizer and Dioctyl Sodium Sulfosuccinate;” U.S. Pat. No. 6,656,504 for “Nanoparticulate Compositions Comprising Amorphous Cyclosporine;” U.S. Pat. No. 6,742,734 for “System and Method for Milling Materials;” U.S. Pat. No. 6,745,962 for “Small Scale Mill and Method Thereof;” U.S. Pat. No. 6,811,767 for “Liquid droplet aerosols of nanoparticulate drugs;” U.S. Pat. No. 6,908,626 for “Compositions having a combination of immediate release and controlled release characteristics;” U.S. Pat. No. 6,969,529 for “Nanoparticulate compositions comprising copolymers of vinyl pyrrolidone and vinyl acetate as surface stabilizers;” U.S. Pat. No. 6,976,647 for “System and Method for Milling Materials;” and U.S. Pat. No. 6,991,191 for “Method of Using a Small Scale Mill;” all of which are specifically incorporated by reference. In addition, U.S. Patent Application No. 20020012675 A1, published on Jan. 31, 2002, for “Controlled Release Nanoparticulate Compositions,” describes nanoparticulate compositions and is specifically incorporated by reference. None of these references describe compositions of nanoparticulate tacrolimus or nanoparticulate sirolimus.

US 20030054042, for “Stabilization of chemical compounds using nanoparticulate formulations,” describes nanoparticulate rapaymcin formulations, including injectable formulations. U.S. Pat. No. 5,989,591 for “Rapamycin formulations for oral administration” describes nanoparticulate rapamycin compositions for oral administration in a tablet dosage form.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter” all of which are specifically incorporated herein by reference.

There is a need for compositions of immunosuppressive agents, such as tacrolimus and sirolimus, that have enhanced solubility characteristics which, in turn, provide enhanced bioavailability upon administration to a patient, as well as reduced fed/fasted absorption variability. The present invention satisfies these needs by providing methods and compositions comprising injectable nanoparticulate formulations of tacrolimus, sirolimus, or a combination thereof. Such injectable nanoparticulate formulations eliminate the need to use solubilizing agents such as polyoxyl 60 hydrogenated castor oil (HCO-60) or a polysorbate, such as polysorbate 80.

### SUMMARY OF THE INVENTION

The invention is directed to an injectable nanoparticulate formulation comprising an immunosuppressive compound, such as tacrolimus, sirolimus, or a combination thereof. The nanoparticulate formulations allow for continuous release from the injection site at a desired rate by altering particle size of the tacrolimus, sirolimus, or a combination thereof. In one embodiment, the formulation is an injectable composition that can be administered subcutaneously or intramuscularly to form a depot that provides long term release of the drug(s). Such a formulation insures better pharmacological efficacy and patient compliance.

The invention also provides an injectable immunosuppressive formulation comprising nanoparticulate tacrolimus, nanoparticulate sirolimus, or combinations thereof, wherein the tacrolimus and/or sirolimus have an effective average particle size of less than about 2000 nm. In addition, the compositions comprise at least one surface stabilizer adsorbed onto or associated with the surface of the tacrolimus and/or sirolimus particles. In other embodiments of the invention, the effective average particle size of the nanoparticulate tacrolimus or sirolimus particles is less than about 1900 nm, less than about 1800 nm, less than about 1700 nm, less than about 1600 nm, less than about 1500 nm, less than about 1400 nm, less than about 1300 nm, less than about 1200 nm, less than about 1250 nm, less than about 1000 nm, less than about 900 nm, less than about 800 nm, less than about 700 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm.

Another aspect of the invention provides for an injectable nanoparticulate tacrolimus, nanoparticulate sirolimus, or a combination tacrolimus/sirolumus formulation that eliminates the need to use polyoxyl 60 hydrogenated castor oil (HCO-60) and/or polysorbate 80 as solubilizers. This is beneficial, as conventional non-nanoparticulate injectable tacrolimus or sirolimus formulations comprise polyoxyl 60 hydrogenated castor oil or polysorbate 80 as solubilizers. The presence of such solubilizing agents can lead to anaphylactic shock (i.e., severe allergic reaction) and death in patients.

The invention also provides for formulations comprising high concentration of tacrolimus, sirolimus, or a combination thereof, in injection volumes to form a depot with slow, long term drug dissolution upon administration.

In another aspect of the invention there is provided a method of preparing injectable nanoparticulate immunosuppressive formulations comprising tacrolimus, sirolimus, or a combination thereof. The method comprises: (1) dispersing the immunosuppressive compound of choice in a liquid dispersion media; and (2) mechanically reducing the particle size of the immunosuppressive compound to a desired effective average particle size, e.g., less than about 2000 nm. One or more surface stabilizers can be added to the composition before, during, or after particle size reduction of the immunosuppressive compound. In one embodiment, the surface stabilizer is a povidone polymer with a molecular weight of less than about 40,000 daltons. Preferably, the liquid dispersion media is maintained at a physiologic pH, for example, within the range of from about 3 to about 8, during the size reduction process.

The invention is also directed to methods of treating a mammal, including a human, using the injectable nanoparticulate formulations of the invention, comprising tacrolimus, sirolimus, or a combination thereof, for the prophylaxis of organ rejection. For example, the compositions are useful in patients receiving allogenic liver or kidney transplants, and for the treatment of psoriasis or other immune diseases. Such methods comprise the step of administering to a subject a therapeutically effective amount of an injectable nanoparticulate formulation of tacrolimus, sirolimus, or a combination thereof, either subcutaneously or intramuscularly so as to form a depot therein for long term administration of the drug.

The injectable nanoparticulate tacrolimus or sirolimus formulation of the invention may optionally include one or more pharmacologically acceptable excipients, such as non-toxic physiologically acceptable liquid carriers, pH adjusting agents, or preservatives.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1. Light micrograph using phase optics at 100× of unmilled tacrolimus.

FIG. 2. Light micrograph using phase optics at 100× of an aqueous dispersion of 10% (w/w) nanoparticulate tacrolimus (Camida LLC) with 2% (w/w) polyvinylpyrrolidone (PVP) K29/32 and 0.05% (w/w) dioctyl sulfosuccinate (DOSS).

FIG. 3: Light micrograph using phase optics at 100× of an aqueous dispersion of 10% (w/w) nanoparticulate tacrolimus (Camida LLC) with 2% (w/w) polyvinylpyrrolidone (PVP) K29/32 and 0.05% (w/w) dioctyl sulfosuccinate (DOSS) following one week of storage under refrigeration.

FIG. 4. Light micrograph using phase optics at 100× of an aqueous dispersion of 10% (w/w) nanoparticulate tacrolimus (Camida LLC), with 2% (w/w) PVP K12 and 0.15% (w/w) sodium deoxycholate.

FIG. 5. Light micrograph using phase optics at 100× of an aqueous dispersion of 20% (w/w) nanoparticulate tacrolimus (Camida LLC), with 3% (w/w) Plasdone® S630 (random copolymer of vinyl pyrrolidone and vinyl acetate in a 60:40 ratio).

FIG. 6. Light micrograph using phase optics at 100× of an aqueous dispersion of 20% (w/w) nanoparticulate tacrolimus (Camida LLC), with 3% (w/w) Plasdone® S630 (random copolymer of vinyl pyrrolidone and vinyl acetate in a 60:40 ratio) following one week of storage under refrigeration.

FIG. 7. Light micrograph using phase optics at 100× of an aqueous dispersion of 10% (w/w) nanoparticulate tacrolimus (Camida LLC), with 2% (w/w) hydroxypropylcellulose (HPC-SL) and 0.1% (w/w) DOSS.

FIG. 8. Light micrograph using phase optics at 100× of an aqueous dispersion of 5% (w/w) nanoparticulate tacrolimus (Camida LLC), with 1% (w/w) HPC-SL and 0.15% (w/w) DOSS.

FIG. 9. Light micrograph using phase optics at 100× of an aqueous dispersion of 5% (w/w) nanoparticulate tacrolimus (Camida LLC), with 1% (w/w) HPC-SL and 0.15% (w/w) DOSS following twelve days of storage under refrigeration.

FIG. 10. Light micrograph using phase optics at 100× of an aqueous dispersion of 5% (w/w) nanoparticulate tacrolimus (Camida LLC), with 1% (w/w) HPC-SL and 0.1% (w/w) sodium deoxycholate.

FIG. 11. Light micrograph using phase optics at 100× of an aqueous dispersion of 5% (w/w) nanoparticulate tacrolimus (Camida LLC), with 1% (w/w) HPC-SL and 0.1% (w/w) sodium deoxycholate following twelve days of storage under refrigeration.

FIG. 12. Light micrograph using phase optics at 100× of an aqueous dispersion of 10% (w/w) nanoparticulate tacrolimus (Camida LLC), with 2% (w/w) hydroxypropylmethyl cellulose (HPMC) and 0.05% (w/w) DOSS.

FIG. 13. Light micrograph using phase optics at 100× of an aqueous dispersion of 10% (w/w) nanoparticulate tacrolimus (Camida LLC), with 2% (w/w) hydroxypropylmethyl cellulose (HPMC) and 0.05% (w/w) DOSS following one week of storage under refrigeration.

FIG. 14. Light micrograph using phase optics at 100× of an aqueous dispersion of 10% (w/w) nanoparticulate tacrolimus (Camida LLC) with 2% Pluronic® F108.

FIG. 15. Light micrograph using phase optics at 100× of an aqueous dispersion of 10% (w/w) nanoparticulate tacrolimus (Camida LLC) with 2% Pluronic® F108 following one week of storage under refrigeration.

FIG. 16. Light micrograph using phase optics at 100× of an aqueous dispersion of 10% (w/w) nanoparticulate tacrolimus (Camida LLC) with 2% Tween® 80.

FIG. 17. Light micrograph using phase optics at 100× of an aqueous dispersion of 10% (w/w) nanoparticulate tacrolimus (Camida LLC) with 2% Tween® 80 following one week of storage under refrigeration.

### DETAILED DESCRIPTION OF THE INVENTION

A. Introduction

The invention is directed to compositions comprising an injectable nanoparticulate immunosuppressive formulation, such as an injectable formulation of nanoparticulate tacrolimus, nanoparticulate sirolimus, or a combination thereof. The immunosuppressant agent utilized in the invention can be any poorly water-soluble immunosuppressant. In one embodiment of the invention, the immunosuppressant agent is tacrolimus, sirolimus or a combination thereof. The nanoparticulate immunosuppressive agent has an effective average particle size of less than about 2000 nm.

Advantages of the formulations of the invention comprising nanoparticulate tacrolimus, nanoparticulate sirolimus, or a combination thereof as compared to conventional, non-nanoparticulate or solubilized forms of tacrolimus or sirolimus include, but are not limited to: (1) increased water solubility; (2) increased bioavailability; (3) smaller dosage form size due to enhanced bioavailability; (4) lower therapeutic dosages due to enhanced bioavailability; (5) reduced risk of unwanted side effects due to lower dosing; (6) enhanced patient convenience and compliance; and (7) more effective prophylaxis of organ rejection after organ replacement surgery or more effective treatment of psoriasis or other immune diseases. A further advantage of the injectable nanoparticulate formulations comprising tacrolimus, sirolimus, or a combination thereof of the invention over conventional forms of injectable tacrolimus or sirolimus is the elimination of the need to use polyoxyl 60 hydrogenated castor oil (HCO-60) or a polysorbate, such as polysorbate 80, as solubilizing agents.

The invention also includes nanoparticulate compositions comprising tacrolimus, sirolimus, or a combination thereof, together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracistenal, intraperitoneal, or topical administration, and the like.

B. Definitions

The invention is described herein using several definitions, as set forth below and throughout the application.

The term “effective average particle size of less than about 2000 nm” as used herein means that at least 50% of the tacrolimus, sirolimus, or tacrolimus and sirolimus particles have a size, by weight, of less than about 2000 nm, when measured by, for example, sedimentation field flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to a stable tacrolimus or sirolimus particle, the term “stable” connotes but is not limited to one or more of the following parameters: (1) tacrolimus or sirolimus particles which do not appreciably flocculate or agglomerate due to interparticle attractive forces or otherwise significantly increase in particle size over time; (2) the physical structure of the tacrolimus or sirolimus particles is not altered over time, such as by conversion from an amorphous phase to a crystalline phase; (3) the tacrolimus or sirolimus particles are chemically stable; and/or (4) where the tacrolimus and/or sirolimus has not been subject to a heating step at or above the melting point of the tacrolimus or sirolimus in the preparation of the nanoparticles of the invention.

The term “conventional” or “non-nanoparticulate” tacrolimus, sirolimus or a combination thereof shall mean an active agent which is solubilized or which has an effective average particle size of greater than about 2000 nm. Nanoparticulate active agents as defined herein have an effective average particle size of less than about 2000 nm.

The phrase “poorly water soluble drugs” as used herein refers drugs having a solubility in water of less than about 30 mg/ml, less than about 20 mg/ml, less than about 10 mg/ml, or less than about 1 mg/ml.

As used herein, the phrase “therapeutically effective amount” shall mean the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “particulate” as used herein refers to a state of matter which is characterized by the presence of discrete particles, pellets, beads or granules irrespective of their size, shape or morphology. The term “multiparticulate” as used herein means a plurality of discrete, or aggregated, particles, pellets, beads, granules or mixture thereof irrespective of their size, shape or morphology.

C. Features of the Nanoparticulate Immunosuppressive Compositions

There are a number of enhanced pharmacological characteristics of the nanoparticulate immunosuppressive compositions of the invention.

1. Increased Bioavailability

The formulations comprising tacrolimus, sirolimus, or a combination thereof of the invention exhibit increased bioavailability at the same dose of the same tacrolimus, sirolimus, or a combination thereof, and require smaller doses as compared to prior conventional tacrolimus or sirolimus formulations.

The non-bioequivalence is significant because it means that the nanoparticulate dosage form of tacrolimus, sirolimus, or combination thereof exhibits significantly greater drug absorption. And for the nanoparticulate dosage form to be bioequivalent to the conventional microcrystalline dosage form, the nanoparticulate dosage form would have to contain significantly less drug. Thus, the nanoparticulate dosage form significantly increases the bioavailability of the drug.

Moreover, a nanoparticulate dosage form comprising tacrolimus, sirolimus, or a combination thereof requires less drug to obtain the same pharmacological effect observed with a conventional microcrystalline dosage form (e.g., PROGRAF®). Therefore, the nanoparticulate dosage form has an increased bioavailability as compared to the conventional microcrystalline dosage form.

2. The Pharmacokinetic Profiles of the Tacrolimus and/or Sirolimus Compositions of the Invention are not Affected by the Fed or Fasted State of the Subject Ingesting the Compositions

The compositions of the invention encompass tacrolimus, sirolimus, or a combination thereof, wherein the pharmacokinetic profile of the tacrolimus, sirolimus, or combination is not substantially affected by the fed or fasted state of a subject ingesting the composition. This means that there is little or no appreciable difference in the quantity of drug absorbed or the rate of drug absorption when the nanoparticulate compositions comprising tacrolimus, sirolimus, or a combination thereof are administered in the fed versus the fasted state.

Benefits of a dosage form which substantially eliminates the effect of food include an increase in subject convenience, thereby increasing subject compliance, as the subject does not need to ensure that they are taking a dose either with or without food. This is significant, as with poor subject compliance with tacrolimus or sirolimus, an increase in the medical condition for which the drug is being prescribed may be observed—e.g., the patient may suffer from organ rejection, or not be treated for psoriasis or other immune diseases

The invention also preferably provides compositions comprising tacrolimus, sirolimus, or a combination thereof having a desirable pharmacokinetic profile when administered to mammalian subjects. The desirable pharmacokinetic profile of the compositions comprising tacrolimus, sirolimus, or a combination thereof preferably includes, but is not limited to: (1) a Cₘₐₓ for tacrolimus, sirolimus, or a combination thereof, when assayed in the plasma of a mammalian subject following administration, that is preferably greater than the Cₘₐₓ for a non-nanoparticulate tacrolimus or sirolimus formulation, administered at the same dosage; and/or (2) an AUC for tacrolimus, sirolimus, or a combination thereof, when assayed in the plasma of a mammalian subject following administration, that is preferably greater than the AUC for a non-nanoparticulate tacrolimus or sirolimus formulation, administered at the same dosage; and/or (3) a Tₘₐₓ for tacrolimus, sirolimus, or a combination thereof, when assayed in the plasma of a mammalian subject following administration, that is preferably less than the Tₘₐₓ for a non-nanoparticulate tacrolimus or sirolimus formulation, administered at the same dosage. The desirable pharmacokinetic profile, as used herein, is the pharmacokinetic profile measured after the initial dose of tacrolimus, sirolimus, or a combination thereof.

In one embodiment, a composition comprising tacrolimus, sirolimus, or a combination thereof exhibits in comparative pharmacokinetic testing with a non-nanoparticulate tacrolimus or sirolimus formulation, administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate tacrolimus or sirolimus formulation.

In another embodiment, the composition comprising tacrolimus, sirolimus, or a combination thereof of the invention exhibits in comparative pharmacokinetic testing with a non-nanoparticulate tacrolimus or sirolimus formulation, administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate tacrolimus or sirolimus formulation.

In yet another embodiment, the composition comprising tacrolimus, sirolimus, or a combination thereof of the invention exhibits in comparative pharmacokinetic testing with a non-nanoparticulate tacrolimus or sirolimus formulation, administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate tacrolimus or sirolimus formulation.

3. Bioequivalency of the Immunosuppressive Compound Containing Compositions of the Invention When Administered in the Fed Versus the Fasted State

The invention also encompasses a composition comprising nanoparticulate tacrolimus, nanoparticulate sirolimus, or a combination thereof in which administration of the composition to a subject in a fasted state is bioequivalent to administration of the composition to a subject in a fed state. The difference in absorption of the compositions comprising the nanoparticulate tacrolimus, nanoparticulate sirolimus, or a combination thereof when administered in the fed versus the fasted state, is preferably less than about 35%, less than about 30%, less than about 25%, less than about 20%, less than about 15%, less than about 10%, less than about 5%, or less than about 3%.

In one embodiment of the invention, the invention encompasses compositions comprising nanoparticulate tacrolimus, nanoparticulate sirolimus, or a combination thereof, wherein administration of the composition to a subject in a fasted state is bioequivalent to administration of the composition to a subject in a fed state, in particular as defined by Cₘₐₓ and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

4. Dissolution Profiles of the Immunosuppressive Compositions of the Invention

The compositions comprising tacrolimus, sirolimus, or a combination thereof of the invention have unexpectedly dramatic dissolution profiles. Rapid dissolution of an administered active agent is preferable, as faster dissolution generally leads to faster onset of action and greater bioavailability. To improve the dissolution profile and bioavailability of comprising tacrolimus, sirolimus, or a combination thereof, it is useful to increase the drug's dissolution so that it could attain a level close to 100%.

The compositions comprising tacrolimus, sirolimus, or a combination thereof of the invention preferably have a dissolution profile in which within about 5 minutes at least about 20% of the composition is dissolved. In other embodiments of the invention, at least about 30% or at least about 40% of the composition comprising tacrolimus, sirolimus, or a combination thereof is dissolved within about 5 minutes. In yet other embodiments of the invention, preferably at least about 40%, at least about 50%, at least about 60%, at least about 70%, or at least about 80% of the composition comprising tacrolimus, sirolimus, or a combination thereof is dissolved within about 10 minutes. Finally, in another embodiment of the invention, preferably at least about 70%, at least about 80%, at least about 90%, or at least about 100% of the composition comprising tacrolimus, sirolimus, or a combination thereof is dissolved within about 20 minutes.

Dissolution is preferably measured in a medium which is discriminating. Such a dissolution medium will produce two very different dissolution curves for two products having very different dissolution profiles in gastric juices, i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

5. Stability of the Immunosuppressive Compositions in Biorelevant Media

An additional feature of the compositions comprising tacrolimus, sirolimus, or a combination thereof of the invention is that the compositions substantially maintain a nanoparticulate particle size when dispersed in a biorelevant media. Biorelevant media mimics conditions found in vivo. As the nanoparticulate active agent compositions of the invention benefit from the small particle size of the active agent; if the active agent does not substantially maintain a nanoparticulate particle size upon administration, then “clumps” or agglomerated active agent particles are formed,. With the formation of such agglomerated particles, the bioavailability of the dosage form may fall.

Preferably, following dispersion in a biorelevant media, the compositions of the invention maintain an effective average particle size of less than about 2000 nm. In other embodiments of the invention, the redispersed tacrolimus and/or sirolimus particles of the invention have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Such methods suitable for measuring effective average particle size are known to a person of ordinary skill in the art.

Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc. Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract. Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

6. Immunosuppressive Compositions Used in Conjunction with Other Active Agents

The compositions comprising tacrolimus, sirolimus, or a combination thereof of the invention can additionally comprise one or more compounds useful in the prophylaxis of organ rejection or treatment of psoriasis or other immune diseases. The compositions of the invention can be co-formulated with such other active agents, or the compositions of the invention can be co-administered or sequentially administered in conjunction with such active agents. Examples of drugs that can be co-administered or co-formulated with tacrolimus and/or sirolimus include, but are not limited to, cyclosporine, mycophenolic acid, alemtuzumab, mycophenolate mofetil, corticosteroids, glucocorticosteroids, doxycycline, interferon beta-1b, malononitrilamide FK778, azathioprine, Campath-1H, basiliximab, and methotrexate.

D. Compositions

The invention provides compositions comprising nanoparticulate tacrolimus, sirolimus, or a combination thereof and at least one surface stabilizer. The surface stabilizers are preferably adsorbed to or associated with the surface of the tacrolimus or sirolimus particles. Surface stabilizers useful herein do not chemically react with the tacrolimus or sirolimus particles or itself. Preferably, individual molecules of the surface stabilizer are essentially free of intermolecular cross-linkages. In another embodiment, the compositions of the invention can comprise two or more surface stabilizers.

The invention also includes nanoparticulate compositions comprising tacrolimus, sirolimus, or a combination thereof together with one or more non-toxic physiologically acceptable carriers, adjuvants, or vehicles, collectively referred to as carriers. The compositions can be formulated for parenteral injection (e.g., intravenous, intramuscular, or subcutaneous), intraperitoneal injection, and the like.

1. Immunosuppressive Active Agent

Exemplary immunosuppressive active agents for use in the injectable dosage forms of the invention are tacrolimus and sirolimus.

Tacrolimus, also known as FK-506 or Fujimycin, is a 23-membered macrolide lactone. As used herein, the term “tacrolimus” includes analogs and salts thereof, and can be in a crystalline phase, an amorphous phase, a semi-crystalline phase, a semi-amorphous phase, or a mixture thereof. Tacrolimus may be present either in the form of one substantially optically pure enantiomer or as a mixture, racemic or otherwise, of enantiomers. Conventional forms of tacrolimus contain solubilizing agents, such as Cremophor®, which are undesirable.

Sirolimus is useful as an immunosuppressant and as an antifungal antibiotic, and its use is described in, for example, U.S. Pat. Nos. 3,929,992, 3,993,749, and 4,316,885, and in Belgian Pat. No. 877,700. The compound, which is only slightly soluble in water, i.e., 20 micrograms per mL, rapidly hydrolyzes when exposed to water. Because sirolimus is highly unstable when exposed to an aqueous medium, special injectable formulations have been developed for administration to patients, such as those described in European Patent No. EP 041,795. Such formulations are often undesirable, as frequently the non-aqueous solubilizing agent exhibits toxic side effects. As used herein, the term “sirolimus” includes analogs and salts thereof, and can be in a crystalline phase, an amorphous phase, a semi-crystalline phase, a semi-amorphous phase, or a mixture thereof. Sirolimus may be present either in the form of one substantially optically pure enantiomer or as a mixture, racemic or otherwise, of enantiomers.

2. Surface Stabilizers

Combinations of more than one surface stabilizer can be used in the injectable formulations comprising tacrolimus, sirolimus or a combination thereof of the invention. Suitable surface stabilizers include, but are not limited to, known organic and inorganic pharmaceutical excipients. Such excipients include various polymers, low molecular weight oligomers, natural products, and surfactants. Surface stabilizers include nonionic, anionic, cationic, ionic, and zwitterionic surfactants. An exemplary surface stabilizer for an injectable nanoparticulate tacrolimus and/or nanoparticulate sirolimus formulation is a povidone polymer.

Representative examples of surface stabilizers include but are not limited to hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxes 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508200 (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-lOG® or Surfactant 10-G® (Olin Chemicals, Stamford, Conn.); Crodestas SL-40® (Croda, Inc.); and SA9OHCO, which is C18H37CH2(CON(CH3)-CH2(CHOH)4(CH20H)2 (Eastman Kodak Co.); decanoyl-N-methylglucamide; n-decyl (-D-glucopyranoside; n-decyl (-D-maltopyranoside; n-dodecyl (-D-glucopyranoside; n-dodecyl (-D-maltoside; heptanoyl-N-methylglucamide; n-heptyl-(-D-glucopyranoside; n-heptyl (-D-thioglucoside; n-hexyl (-D-glucopyranoside; nonanoyl-N-methylglucamide; n-noyl (-D-glucopyranoside; octanoyl-N-methylglucamide; n-octyl-(-D-glucopyranoside; octyl (-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate. Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C12-15dimethyl hydroxyethyl ammonium chloride or bromide, coconut dimethyl hydroxyethyl ammonium chloride or bromide, myristyl trimethyl ammonium methyl sulfate, lauryl dimethyl benzyl ammonium chloride or bromide, lauryl dimethyl (ethenoxy)4 ammonium chloride or bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C12-14) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12, C15, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336), POLYQUAT, tetrabutylammonium bromide, benzyl trimethylammonium bromide, choline esters (such as choline esters of fatty acids), benzalkonium chloride, stearalkonium chloride compounds (such as stearyltrimonium chloride and distearyldimonium chloride), cetyl pyridinium bromide or chloride, halide salts of quaternized polyoxyethylalkylamines, MIRAPOL and ALKAQUAT (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR1R2R3R4(+). For compounds of the formula NR1R2R3R4(+):

(i) none of R1-R4 are CH3;

(ii) one of R1-R4 is CH3;

(iii) three of R1-R4 are CH3;

(iv) all of R1-R4 are CH3;

(v) two of R1-R4 are CH3, one of R1-R4 is C6H5CH2, and one of R1-R4 is an alkyl chain of seven carbon atoms or less;

(vi) two of R1-R4 are CH3, one of R1-R4 is C6H5CH2, and one of R1-R4 is an alkyl chain of nineteen carbon atoms or more;

(vii) two of R1-R4 are CH3 and one of R1-R4 is the group C6H5(CH2)n, where n>1;

(viii) two of R1-R4 are CH3, one of R1-R4 is C6H5CH2, and one of R1-R4 comprises at least one heteroatom;

(ix) two of R1-R4 are CH3, one of R1-R4 is C6H5CH2, and one of R1-R4 comprises at least one halogen;

(x) two of R1-R4 are CH3, one of R1-R4 is C6H5CH2, and one of R1-R4 comprises at least one cyclic fragment;

(xi) two of R1-R4 are CH3 and one of R1-R4 is a phenyl ring; or

(xii) two of R1-R4 are CH3 and two of R1-R4 are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quatemium-15), distearyldimonium chloride (Quatemium-5), dodecyl dimethyl ethylbenzyl ammonium chloride (Quaternium-14), Quaternium-22, Quatemium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated herein by reference.

Povidone Polymers

Povidone polymers are exemplary surface stabilizers for use in formulating an injectable nanoparticulate tacrolimus and/or nanoparticulate sirolimus formulation. Povidone polymers, also known as polyvidon(e), povidonum, PVP, and polyvinylpyrrolidone, are sold under the trade names Kollidon® (BASF Corp.) and Plasdone® (ISP Technologies, Inc.). They are polydisperse macromolecular molecules, with a chemical name of 1-ethenyl-2-pyrrolidinone polymers and 1-vinyl-2-pyrrolidinone polymers. Povidone polymers are produced commercially as a series of products having mean molecular weights ranging from about 10,000 to about 700,000 daltons. To be useful as a surface modifier for a drug compound to be administered to a mammal, the povidone polymer must have a molecular weight of less than about 40,000 daltons, as a molecular weight of greater than 40,000 daltons would have difficulty clearing the body.

Povidone polymers are prepared by, for example, Reppe's process, comprising: (1) obtaining 1,4-butanediol from acetylene and formaldehyde by the Reppe butadiene synthesis; (2) dehydrogenating the 1,4-butanediol over copper at 200° to form γ-butyrolactone; and (3) reacting γ-butyrolactone with ammonia to yield pyrrolidone. Subsequent treatment with acetylene gives the vinyl pyrrolidone monomer. Polymerization is carried out by heating in the presence of H₂O and NH₃. See The Merck Index, 10^{th} Edition, pp. 7581 (Merck & Co., Rahway, N.J., 1983).

The manufacturing process for povidone polymers produces polymers containing molecules of unequal chain length, and thus different molecular weights. The molecular weights of the molecules vary about a mean or average for each particular commercially available grade. Because it is difficult to determine the polymer's molecular weight directly, the most widely used method of classifying various molecular weight grades is by K-values, based on viscosity measurements. The K-values of various grades of povidone polymers represent a function of the average molecular weight, and are derived from viscosity measurements and calculated according to Fikentscher's formula.

The weight-average of the molecular weight, Mw, is determined by methods that measure the weights of the individual molecules, such as by light scattering. Table 1 provides molecular weight data for several commercially available povidone polymers, all of which are soluble.

TABLE 1

| Povidone | K-Value | Mv (Daltons)** | Mw (Daltons)** | Mn (Daltons)** |
|---|---|---|---|---|
| Plasdone C-15 ® | 17 ± 1 | 7,000 | 10,500 | 3,000 |
| Plasdone C-30 ® | 30.5 ± 1.5 | 38,000 | 62,500* | 16,500 |
| Kollidon 12 PF ® | 11-14 | 3,900 | 2,000-3,000 | 1,300 |
| Kollidon 17 PF ® | 16-18 | 9,300 | 7,000-11,000 | 2,500 |
| Kollidon 25 ® | 24-32 | 25,700 | 28,000-34,000 | 6,000 |

*Because the molecular weight is greater than 40,000 daltons, this povidone polymer is not useful as a surface stabilizer for a drug compound to be administered parenterally (i.e., injected).
**Mv is the viscosity-average molecular weight, Mn is the number-average molecular weight, and Mw is the weight average molecular weight. Mw and Mn were determined by light scattering and ultra-centrifugation, and Mv was determined by viscosity measurements.

Based on the data provided in Table 1, exemplary useful commercially available povidone polymers include, but are not limited to, Plasdone C-15®, Kollidon 12 PF®, Kollidon 17 PF®, and Kollidon 25®.

3. Nanoparticulate Tacrolimus and Sirolimus Particle Size

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

The immunosuppressive compositions of the invention comprise tacrolimus and/or sirolimus nanoparticles having an effective average particle size of less than about 2000 nm (i.e., 2 microns). In other embodiments of the invention, the tacrolimus and sirolimus nanoparticles have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

An “effective average particle size of less than about 2000 nm” means that at least 50% of the tacrolimus, sirolimus, or tacrolimus and sirolimus particles have a particle size less than the effective average, by weight, i.e., less than about 2000 nm. If the “effective average particle size” is less than about 1900 nm, then at least about 50% of the tacrolimus, sirolimus, or tacrolimus and sirolimus particles have a size of less than about 1900 nm, when measured by the above-noted techniques. The same is true for the other particle sizes referenced above. In other embodiments, at least about 60%, at least about 70%, at least about 80%, at least about 90%, at least about 95%, or at least about 99% of the tacrolimus, sirolimus, or tacrolimus and sirolimus particles have a particle size less than the effective average, i.e., less than about 2000 nm, less than about 1900 nm, less than about 1800 nm, etc.

In the invention, the value for D50 of a nanoparticulate tacrolimus, sirolimus, or tacrolimus and sirolimus composition is the particle size below which 50% of the tacrolimus, sirolimus, or tacrolimus and sirolimus particles fall, by weight. Similarly, D90 is the particle size below which 90% of the tacrolimus, sirolimus, or tacrolimus and sirolimus particles fall, by weight.

4. Concentration of Nanoparticulate Immunosuppressive Compound and Surface Stabilizers

The relative amounts of tacrolimus, sirolimus and combination thereof and one or more surface stabilizers can vary widely. The optimal amount of the individual components depends, for example, upon physical and chemical attributes of the surface stabilizer(s) selected, such as the hydrophilic lipophilic balance (HLB), melting point, and the surface tension of water solutions of the stabilizer, etc.

Preferably, the concentration of tacrolimus, sirolimus, or combination thereof can vary from about 99.5% to about 0.001%, from about 95% to about 0.1%, or from about 90% to about 0.5%, by weight, based on the total combined weight of the tacrolimus, sirolimus or combination thereof and at least one surface stabilizer, not including other excipients. Higher concentrations of the active ingredient are generally preferred from a dose and cost efficiency standpoint.

Preferably, the concentration of surface stabilizer can vary from about 0.5% to about 99.999%, from about 5.0% to about 99.9%, or from about 10% to about 99.5%, by weight, based on the total combined dry weight of active agent and at least one surface stabilizer, not including other excipients.

5. Other Pharmaceutical Excipients

Pharmaceutical compositions of the invention may also comprise one or more binding agents, filling agents, lubricating agents, suspending agents, sweeteners, flavoring agents, preservatives, buffers, wetting agents, disintegrants, effervescent agents, and other excipients depending upon the route of administration and the dosage form desired. Such excipients are well known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCCυ).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, and quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples, such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

6. Injectable Nanoparticulate Tacrolimus Formulations

The invention provides injectable nanoparticulate formulations comprising tacrolimus, sirolimus or a combination thereof that can comprise high drug concentrations in low injection volumes. Exemplary formulations comprise, based on % w/w:

| | |
|---|---|
| Immunosuppressant active | 1.0-50% |
| Surface stabilizer | 0.1-50% |
| Preservatives | 0.05-0.25% |
| pH adjusting agent | pH about 6 to about 7 |
| Water q.s. | |

Exemplary preservatives include methylparaben (about 0.18% based on % w/w), propylparaben (about 0.02% based on % w/w), phenol (about 0.5% based on % w/w), and benzyl alcohol (up to 2% v/v). An exemplary pH adjusting agent is sodium hydroxide, and an exemplary liquid carrier is sterile water for injection. Other useful preservatives, pH adjusting agents, and liquid carriers are well-known in the art.

The tacrolimus or sirolimus active in the invention may be present either in the form of one substantially optically pure enantiomer or as a mixture, racemic or otherwise, of enantiomers. The immunosuppressant agent is preferably present in an injectable nanoparticulate formulation of the invention in an amount of from about 0.01 mg to about 50 mg, or in an amount of from about 0.05 mg to about 20 mg.

E. Methods of Making Nanoparticulate Tacrolimus and/or Sirolimus Formulations

Nanoparticulate tacrolimus and/or sirolimus compositions can be made using any suitable method known in the art such as, for example, milling, homogenization, precipitation, or supercritical fluid particle generation techniques. Exemplary methods of making nanoparticulate active agent compositions are described in U.S. Pat. No. 5,145,684. Methods of making nanoparticulate active agent compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Patent No. 5,470,583 for “Method of Preparing Nanoparticle Compositions Containing Charged Phospholipids to Reduce Aggregation,” all of which are specifically incorporated herein by reference.

The resultant nanoparticulate tacrolimus and/or sirolimus compositions or dispersions can be utilized in solid, semi-solid, or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc. In the present invention, injectable dosage forms are preferred.

In another aspect of the invention there is provided a method of preparing the injectable nanoparticulate immunosuppressant formulations of the invention. The method comprises the steps of: (1) dispersing the desired dosage amount of tacrolimus, sirolimus or a combination thereof in a liquid dispersion medium; and (2) mechanically reducing the particle size of the tacrolimus, sirolimus or combination thereof to an effective average particle size of less than about 2000 nm. A surface stabilizer can be added to the dispersion media either before, during, or after particle size reduction of the active agent. In one embodiment, the surface stabilizer is a povidone polymer having a molecular weight of less than about 40,000 daltons. The liquid dispersion medium can be maintained at a physiologic pH, for example, within the range of from about 3.0 to about 8.0 during the size reduction process; more preferably within the range of from about 5.0 to about 7.5 during the size reduction process. In another embodiment, the dispersion medium used for the size reduction process is aqueous.

Using a particle size reduction method, the particle size of the immunosuppressant is reduced to an effective average particle size of less than about 2000 nm. Effective methods of providing mechanical force for particle size reduction of the tacrolimus or sirolimus immunosuppressant include ball milling, media milling, and homogenization, for example, with a Microfluidizer® (Microfluidics Corp.). Ball milling is a low energy milling process that uses milling media, drug, stabilizer, and liquid. The materials are placed in a milling vessel that is rotated at optimal speed such that the media cascades and reduces the drug particle size by impaction. The media used must have a high density as the energy for the particle reduction is provided by gravity and the mass of the attrition media.

Media milling is a high energy milling process. Drug, stabilizer, and liquid are placed in a reservoir and re-circulated in a chamber containing media and a rotating shaft/impeller. The rotating shaft agitates the media which subjects the drug to impaction and sheer forces, thereby reducing the drug particle size.

Homogenization is a technique that does not use milling media. Drug, stabilizer, and liquid (or drug and liquid with the stabilizer added after particle size reduction) constitute a process stream propelled into a process zone, which in the Microfluidizer® is called the Interaction Chamber. The product to be treated is inducted into the pump, and then forced out. The priming valve of the Microfluidizer® purges air out of the pump. Once the pump is filled with product, the priming valve is closed and the product is forced through the interaction chamber. The geometry of the interaction chamber produces powerful forces of sheer, impact, and cavitation which are responsible for particle size reduction. Specifically, inside the interaction chamber, the pressurized product is split into two streams and accelerated to extremely high velocities. The formed jets are then directed toward each other and collide in the interaction zone. The resulting product has very fine and uniform particle or droplet size. The Microfluidizer® also provides a heat exchanger to allow cooling of the product. U.S. Pat. No. 5,510,118, which is specifically incorporated by reference, refers to a process using a Microfluidizer®.

The immunosuppressant can be added to a liquid medium in which it is essentially insoluble to form a premix. The surface stabilizer can be present in the premix or it can be added to the drug dispersion following particle size reduction. The premix can be used directly by subjecting it to mechanical means to reduce the average tacrolimus or sirolimus particle size in the dispersion to less than about 2000 nm. It is preferred that the premix be used directly when a ball mill is used for attrition. Alternatively, the immunosuppressant and at least one surface stabilizer can be dispersed in the liquid medium using suitable agitation, e.g., a Cowles type mixer, until a homogeneous dispersion is observed in which there are no large agglomerates visible to the naked eye. It is preferred that the premix be subjected to such a pre-milling dispersion step when a re-circulating media mill is used for attrition.

The mechanical means applied to reduce the tacrolimus or sirolimus particle size can take the form of a dispersion mill. Suitable dispersion mills include a ball mill, an attritor mill, a vibratory mill, and media mills such as a sand mill and a bead mill. A media mill is preferred due to the relatively shorter milling time required to provide the desired reduction in particle size. For media milling, the apparent viscosity of the premix is preferably from about 100 to about 1000 centipoise, and for ball milling the apparent viscosity of the premix is preferably from about 1 up to about 100 centipoise. Such ranges tend to afford an optimal balance between efficient particle size reduction and media erosion.

The attrition time can vary widely and depends primarily upon the particular mechanical means and processing conditions selected. For ball mills, processing times of up to five days or longer may be required. Alternatively, processing times of less than 1 day (residence times of one minute up to several hours) are possible with the use of a high shear media mill.

The tacrolimus or sirolimus particles can be reduced in size at a temperature which does not significantly degrade the immunosuppressant molecule. Processing temperatures of less than about 30 to less than about 40° C. are ordinarily preferred. If desired, the processing equipment can be cooled with conventional cooling equipment. Control of the temperature, e.g., by jacketing or immersion of the milling chamber in ice water, is contemplated. Generally, the method of the invention is conveniently carried out under conditions of ambient temperature and at processing pressures which are safe and effective for the milling process. Ambient processing pressures are typical of ball mills, attritor mills, and vibratory mills.

Grinding Media

The grinding media for the particle size reduction step can be selected from rigid media preferably spherical or particulate in form having an average size less than about 3 mm and, more preferably, less than about 1 mm. Such media desirably can provide the particles of the invention with shorter processing times and impart less wear to the milling equipment. The selection of material for the grinding media is not believed to be critical. Zirconium oxide, such as 95% ZrO stabilized with magnesia, zirconium silicate, ceramic, stainless steel, titania, alumina, 95% ZrO stabilized with yttrium, glass grinding media, and polymeric grinding media are exemplary grinding materials.

The grinding media can comprise particles that are preferably substantially spherical in shape, e.g., beads, consisting essentially of polymeric resin or other suitable material. Alternatively, the grinding media can comprise a core having a coating of a polymeric resin adhered thereon. The polymeric resin can have a density from about 0.8 to about 3.0 g/cm³.

In general, suitable polymeric resins are chemically and physically inert, substantially free of metals, solvent, and monomers, and of sufficient hardness and friability to enable them to avoid being chipped or crushed during grinding. Suitable polymeric resins include crosslinked polystyrenes, such as polystyrene crosslinked with divinylbenzene; styrene copolymers; polycarbonates; polyacetals, such as Delrin® (E.I. du Pont de Nemours and Co.); vinyl chloride polymers and copolymers; polyurethanes; polyamides; poly(tetrafluoroethylenes), e.g., Teflon® (E.I. du Pont de Nemours and Co.), and other fluoropolymers; high density polyethylenes; polypropylenes; cellulose ethers and esters such as cellulose acetate; polyhydroxymethacrylate; polyhydroxyethyl acrylate; and silicone-containing polymers such as polysiloxanes and the like. The polymer can be biodegradable. Exemplary biodegradable polymers include poly(lactides), poly(glycolide) copolymers of lactides and glycolide, polyanhydrides, poly(hydroxyethyl methacylate), poly(imino carbonates), poly(N-acylhydroxyproline)esters, poly(N-palmitoyl hydroxyproline) esters, ethylene-vinyl acetate copolymers, poly(orthoesters), poly(caprolactones), and poly(phosphazenes). For biodegradable polymers, contamination from the media itself advantageously can metabolize in vivo into biologically acceptable products that can be eliminated from the body.

The grinding media preferably ranges in size from about 0.01 to about 3 mm. For fine grinding, the grinding media is preferably from about 0.02 to about 2 mm, and more preferably from about 0.03 to about 1 mm in size.

In a preferred grinding process the particles are made continuously. Such a method comprises continuously introducing the tacrolimus or sirolimus active into a milling chamber, contacting the compounds with grinding media while in the chamber to reduce the particle size, and continuously removing the nanoparticulate active from the milling chamber.

The grinding media is separated from the milled nanoparticulate tacrolimus or sirolimus using conventional separation techniques, in a secondary process such as by simple filtration, sieving through a mesh filter or screen, and the like. Other separation techniques such as centrifugation may also be employed.

Sterile Product Manufacturing

Development of injectable compositions requires the production of a sterile product. The manufacturing process of the present invention is similar to typical known manufacturing processes for sterile suspensions. A typical sterile suspension manufacturing process flowchart is as follows:

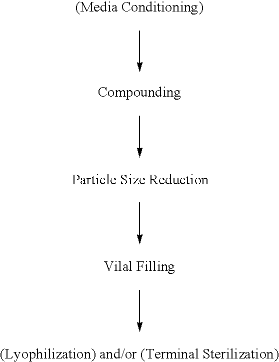

As indicated by the optional steps in parentheses, some of the processing is dependent upon the method of particle size reduction and/or method of sterilization. For example, media conditioning is not required for a milling method that does not use media. If terminal sterilization is not feasible due to chemical and/or physical instability, aseptic processing can be used.

F. Method of Treatment

Yet another aspect of the present invention provides a method of treating a mammal, including a human, using the injectable nanoparticulate tacrolimus or sirolimus formulations of the invention for the prophylaxis of organ rejection or treatment of psoriasis or other immune diseases. Such methods comprise the step of administering to a subject a therapeutically effective amount of the injectable nanoparticulate tacrolimus or sirolimus formulations of the invention so as to form a subcutaneous or intra-muscular depot within the patient. The depot slowly releases the active over time to provide long term treatment to the allogenic organ recipient or treatment of psoriasis or other immune diseases. The depot formulations of tacrolimus or sirolimus can provide immunosuppressant therapy for up to a year if so required.

In other embodiments of the invention, the injectable depot nanoparticulate tacrolimus, sirolimus, or a combination thereof composition provides therapeutic levels of drug for up to about 1 week, up to about 2 weeks, up to about 3 weeks, up about 4 weeks, up to about 5 weeks, up to about 6 weeks, up to about 7 weeks, up to about 8 weeks, up to about 9 weeks, up to about 10 weeks, up to about 11 weeks, up to about 12 weeks, up to about 1 month, up to about 2 months, up to about 3 months, up to about 4 months, up to about 5 months, up to about 6 months, up to about 7 months, up to about 8 months, up to about 9 months, up to about 10 months, up to about 11 months, or up to about 1 year.

A particularly advantageous feature of the invention is that the injectable nanoparticulate tacrolimus, sirolimus, or tacrolimus and sirolimus formulations of the invention can be injected into the patient as a depot and yet eliminate the need to use polyoxyl 60 hydrogenated castor oil (HCO-60) and/or a polysorbate, such as polysorbate 80, as solubilizers. In addition, the injectable formulations of the invention can provide a high concentration of tacrolimus, sirolimus, or combination thereof in a depot delivery system for long term therapeutic efficacy.

One of ordinary skill will appreciate that effective amounts of tacrolimus sirolimus, or a combination thereof can be determined empirically and can be employed in pure form or, where such forms exist, in pharmaceutically acceptable salt, ester, or prodrug form. The selected dosage level therefore depends upon the desired therapeutic effect, the route of administration, the potency of the administered tacrolimus, sirolimus, or combination thereof, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following examples are given to illustrate the invention. It should be understood, however, that the spirit and scope of the invention is not to be limited to the specific conditions or details described in these examples but should only be limited by the scope of the claims that follow. All references identified herein, including U.S. patents, are hereby expressly incorporated by reference.

### EXAMPLES

### Example 1

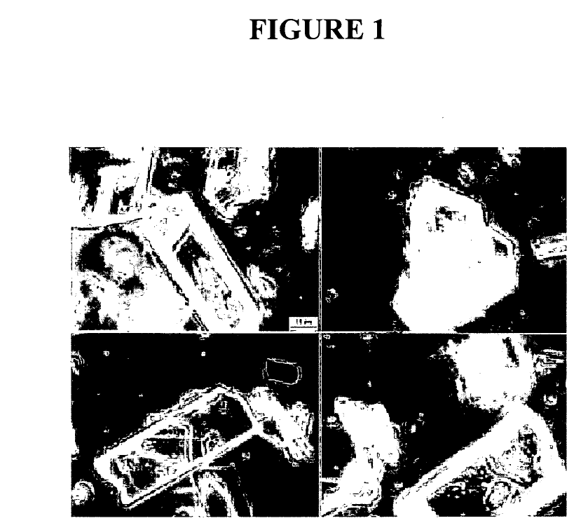

The purpose of this example was to prepare a nanoparticulate tacrolimus formulation suitable for use as an injectable dosage form. FIG. 1 shows a light micrograph using phase optics at 100× of unmilled tacrolimus.

An aqueous dispersion of 10% (w/w) tacrolimus (Camida LLC), combined with 2% (w/w) polyvinylpyrrolidone (PVP) K29/32 and 0.05% (w/w) dioctylsulfosuccinate (DOSS), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 minutes.

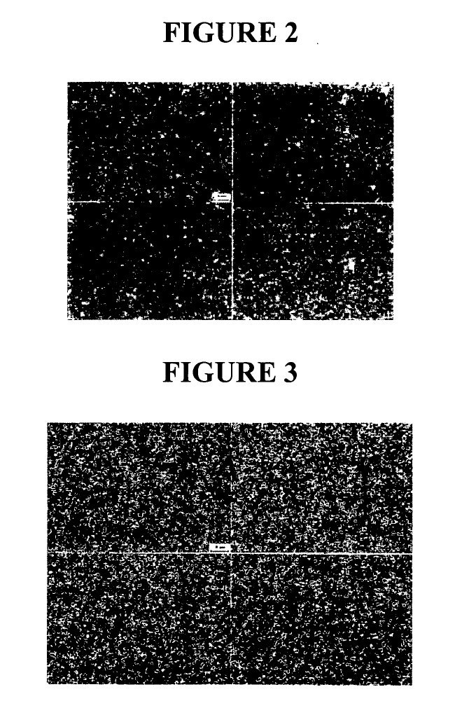

Following milling, the particle size of the milled tacrolimus particles was measured, in deionized distilled water, using a Horiba LA 910 particle size analyzer. The initial mean milled tacrolimus particle size was 192 nm, with a D50 of 177 nm and a D90 of 278 nm, as shown in Table 1. FIG. 2 shows a light micrograph using phase optics at 100× of the milled tacrolimus. In a second measurement in distilled water following 1 week of refrigeration at <15° C., the mean tacrolimus particle size was 245 nm, with a D50 of 219 nm and a D90 of 374 nm. FIG. 3 shows a light micrograph using phase optics at 100× of the milled tacrolimus following one week of refrigeration.

TABLE 1

| | Mean | D50 | D90 |
|---|---|---|---|
| | Particle | Particle | Particle |
| Sample | Size (nm) | Size (nm) | Size (nm) |
| initial tacrolimus/PVP/DOSS | 192 | 177 | 278 |
| sample | | | |
| tacrolimus/PVP/DOSS sample | 245 | 219 | 374 |
| following 1 week refrigeration | | | |

The results demonstrate the successful preparation of a stable nanoparticulate tacrolimus formulation, as the mean particle size obtained was 192 nm, and minimal particle size growth was observed following storage.

### Example 2

The purpose of this example was to prepare a nanoparticulate tacrolimus formulation suitable for use as an injectable dosage form.

An aqueous dispersion of 10% (w/w) tacrolimus (Camida LLC), combined with 2% PVP K12 and 0.15% sodium deoxycholate, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 150 minutes.

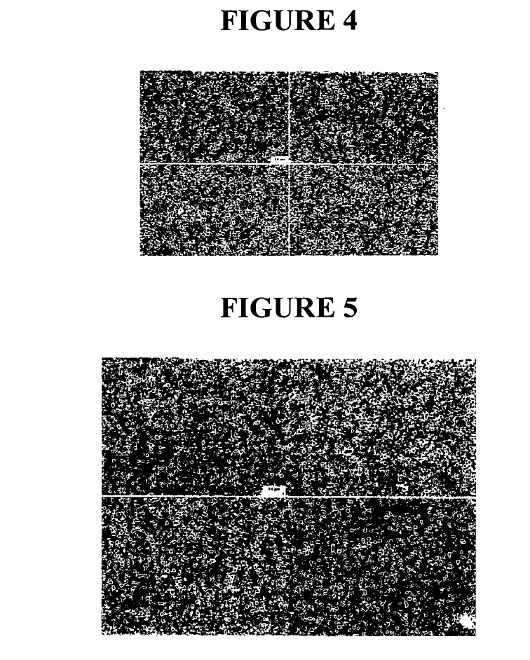

Following milling, the particle size of the milled tacrolimus particles was measured, in deionized distilled water, using a Horiba LA 910 particle size analyzer. The mean milled tacrolimus particle size was 329 nm, with a D50 of 303 nm and a D90 of 466 nm. FIG. 4 shows a light micrograph using phase optics at 100× of the milled tacrolimus.

The results demonstrate the successful preparation of a stable nanoparticulate tacrolimus formulation, as the mean particle size obtained was 329 nm.

### Example 3

The purpose of this example was to prepare a nanoparticulate tacrolimus formulation suitable for use as an injectable dosage form.

An aqueous dispersion of 20% (w/w) tacrolimus (Camida LLC), combined with 3% (w/w) Pluronic® S630 and 0.05% (w/w) DOSS, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 minutes. A light micrograph using phase optics at 100× of the milled tacrolimus is shown in FIG. 5.

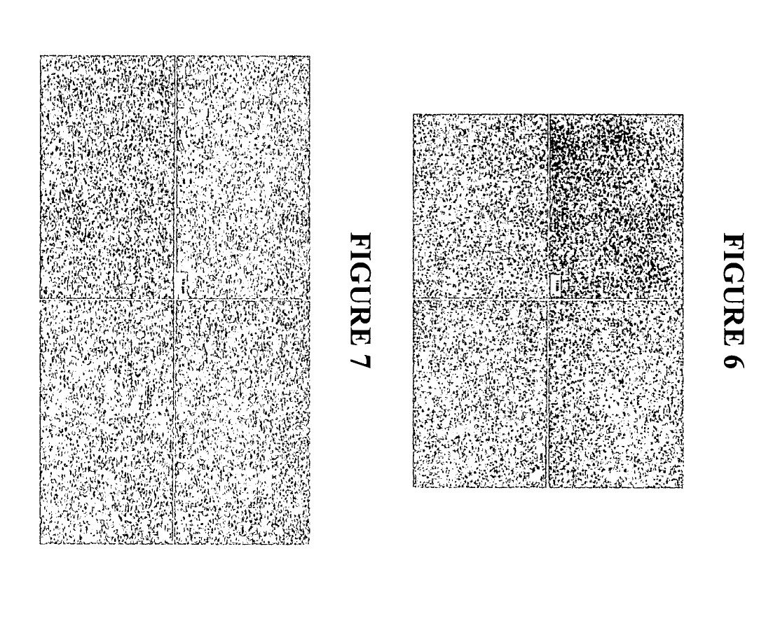

Following milling, the particle size of the milled tacrolimus particles was measured, in deionized distilled water, using a Horiba LA 910 particle size analyzer. The initial mean milled tacrolimus particle size was 171 nm, with a D50 of 163 nm and a D90 of 230 nm, as shown below in Table 2. In a second measurement in distilled water following 1 week of refrigeration at <15° C., the mean tacrolimus particle size was 194 nm, with a D50 of 180 nm and a D90 of 279 nm. A light micrograph using phase optics at 100× of the milled tacrolimus following one week of storage under refrigeration is shown in FIG. 6.

TABLE 2

| | Mean | D50 | D90 |
|---|---|---|---|
| | Particle | Particle | Particle |
| Sample | Size (nm) | Size (nm) | Size (nm) |
| initial tacrolimus/Pluronic ® | 171 | 163 | 230 |
| S630/DOSS sample | | | |
| tacrolimus/Pluronic ® S630/DOSS | 194 | 180 | 279 |
| sample following 1 week | | | |
| refrigeration | | | |

The results demonstrate the successful preparation of a stable nanoparticulate tacrolimus formulation, as the mean particle size obtained was 171 nm, and minimal particle size growth was observed following storage.

### Example 4

The purpose of this example was to prepare a nanoparticulate tacrolimus formulation suitable for use as an injectable dosage form.

An aqueous dispersion of 10% (w/w) tacrolimus (Camida LLC), combined with 2% (w/w) hydroxypropylcellulose (HPC-SL) and 0.1% (w/w) DOSS, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 150 minutes. A light micrograph using phase optics at 100× of the milled tacrolimus is shown in FIG. 7.

Following milling, the particle size of the milled tacrolimus particles was measured, in deionized distilled water, using a Horiba LA 910 particle size analyzer. The mean milled tacrolimus particle size was 389 nm, with a D50 of 328 nm and a D90 of 614 nm.

The results demonstrate the successful preparation of a stable nanoparticulate tacrolimus formulation, as the mean particle size obtained was 389 nm.

### Example 5

The purpose of this example was to prepare a nanoparticulate tacrolimus formulation suitable for use as an injectable dosage form.

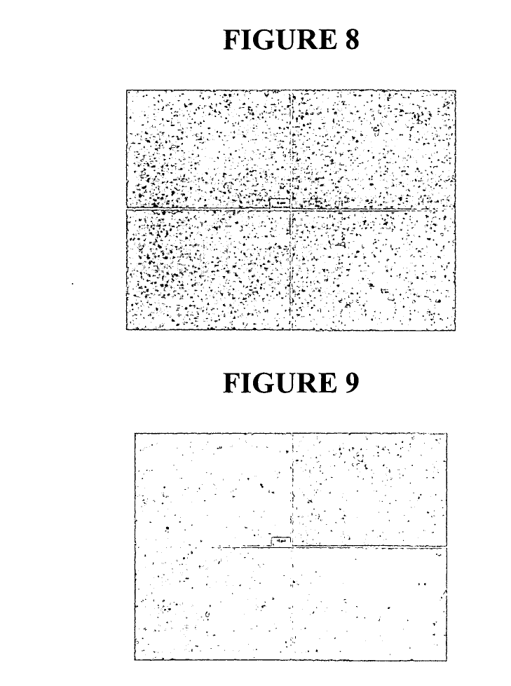

An aqueous dispersion of 5% (w/w) tacrolimus (Camida LLC), combined with 1% (w/w) HPC-SL and 0.15% (w/w) DOSS, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 5500 rpms for 90 minutes. A light micrograph using phase optics at 100× of the milled tacrolimus is shown in FIG. 8.

Following milling, the particle size of the milled tacrolimus particles was measured, in deionized distilled water, using a Horiba LA 910 particle size analyzer. The initial mean milled tacrolimus particle size was 169 nm, with a D50 of 160 nm and a D90 of 225 nm, as shown below in Table 3. In a second measurement in distilled water following 12 days of refrigeration at <15° C., the mean tacrolimus particle size was 155 nm, with a D50 of 138 nm and a D90 of 216 nm. A light micrograph using phase optics at 100× of the milled tacrolimus following twelve days of storage under refrigeration is shown in FIG. 9.

TABLE 3

| | Mean | D50 | D90 |
|---|---|---|---|
| | Particle | Particle | Particle |
| Sample | Size (nm) | Size (nm) | Size (nm) |
| initial tacrolimus/HPC-SL/DOSS | 169 | 160 | 225 |
| sample | | | |
| tacrolimus/HPC-SL/DOSS sample | 155 | 138 | 216 |
| following 12 days refrigeration | | | |

The results demonstrate the successful preparation of a stable nanoparticulate tacrolimus formulation, as the mean particle size obtained was 169 nm, and minimal change in particle size was observed following storage.

### Example 6

The purpose of this example was to prepare a nanoparticulate tacrolimus formulation suitable for use as an injectable dosage form.

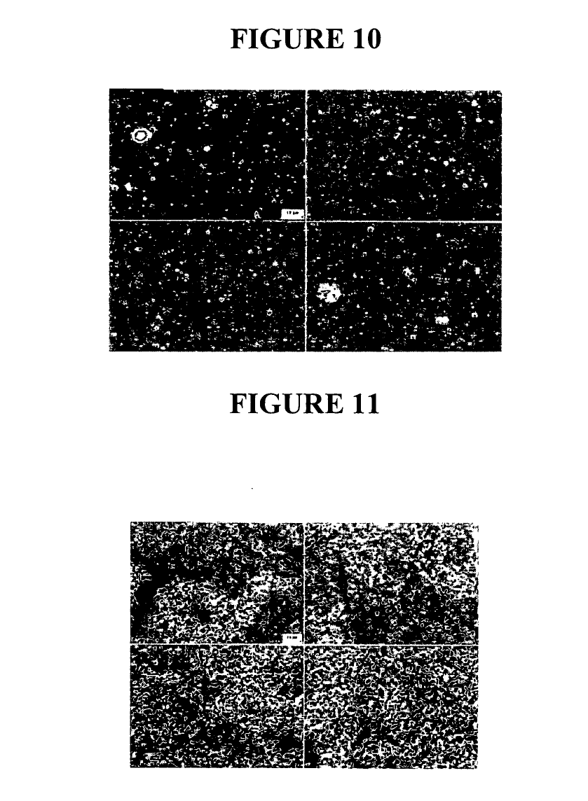

An aqueous dispersion of 5% (w/w) tacrolimus (Camida LLC), combined with 1% (w/w) HPC-SL and 0.1% (w/w) sodium deoxycholate, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 5500 rpms for 75 minutes. A light micrograph using phase optics at 100× of the milled tacrolimus is shown in FIG. 10.

Following milling, the particle size of the milled tacrolimus particles was measured, in deionized distilled water, using a Horiba LA 910 particle size analyzer. The initial mean milled tacrolimus particle size was 1,780 nm, with a D50 of 220 nm and a D90 of 6,665 nm, as shown below in Table 4. In a second measurement in distilled water following 12 days of refrigeration at <15° C., the mean tacrolimus particle size was 65,100 nm, with a D50 of 31,252 nm and a D90 of 175,813 nm. A light micrograph using phase optics at 100× of the milled tacrolimus following twelve days of storage under refrigeration is shown in FIG. 11.

TABLE 4

| | Mean | D50 | D90 |
|---|---|---|---|
| | Particle | Particle | Particle |
| Sample | Size (nm) | Size (nm) | Size (nm) |
| initial tacrolimus/HPC-SL/sodium | 1780 | 220 | 6665 |
| deoxycholate | | | |
| tacrolimus/HPC-SL/sodium | 65,100 | 31,252 | 175,813 |
| deoxycholate sample following 12 | | | |
| days refrigeration | | | |

The results demonstrate the unsuccessful preparation of a stable nanoparticulate tacrolimus formulation, as significant particle size growth and agglomeration were observed following twelve days of storage. Moreover, the light micrograph using phase optics at 100× following milling also shows the presence of large, possible “unmilled” crystals.

### Example 7

The purpose of this example was to prepare a nanoparticulate tacrolimus formulation suitable for use as an injectable dosage form.

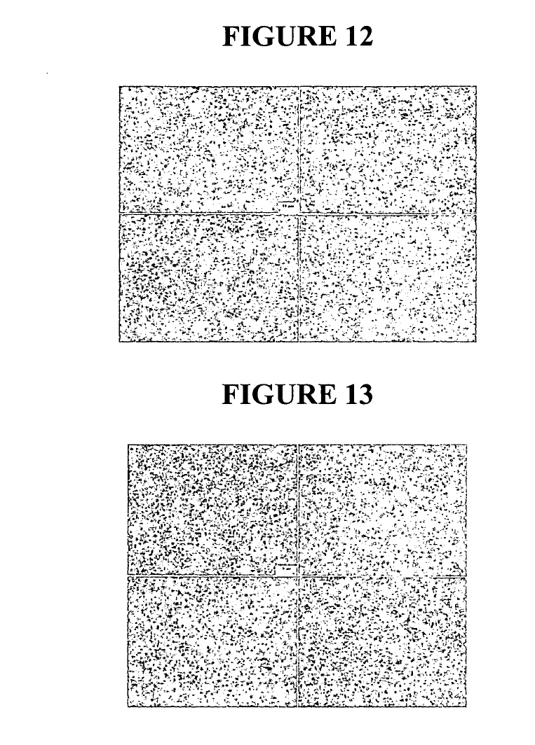

An aqueous dispersion of 10% (w/w) tacrolimus (Camida LLC) combined with 2% (w/w) hydroxypropylmethylcellulose (HPMC) and 0.05% (w/w) DOSS, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 minutes. A light micrograph using phase optics at 100× of the milled tacrolimus is shown in FIG. 12.

Following milling, the particle size of the milled tacrolimus particles was measured, in deionized distilled water, using a Horiba LA 910 particle size analyzer. The initial mean milled tacrolimus particle size was 215 nm, with a D50 of 196 nm and a D90 of 311 nm, as shown below in Table 5. In a second measurement in distilled water following 1 week of refrigeration at <15° C., the mean tacrolimus particle size was 227 nm, with a D50 of 206 nm and a D90 of 337 nm. A light micrograph using phase optics at 100× of the milled tacrolimus following one week of storage under refrigeration is shown in FIG. 13.

TABLE 5

| | Mean | D50 | D90 |
|---|---|---|---|
| | Particle | Particle | Particle |
| Sample | Size (nm) | Size (nm) | Size (nm) |
| initial tacrolimus/HPMC/DOSS | 215 | 196 | 311 |
| tacrolimus/HPMC/DOSS sample | 227 | 206 | 337 |
| following 1 week refrigeration | | | |

The results demonstrate the successful preparation of a stable nanoparticulate tacrolimus formulation, as the mean particle size obtained was 215 nm, and minimal particle size growth was observed following storage.

### Example 8

The purpose of this example was to prepare a nanoparticulate tacrolimus formulation suitable for use as an injectable dosage form.

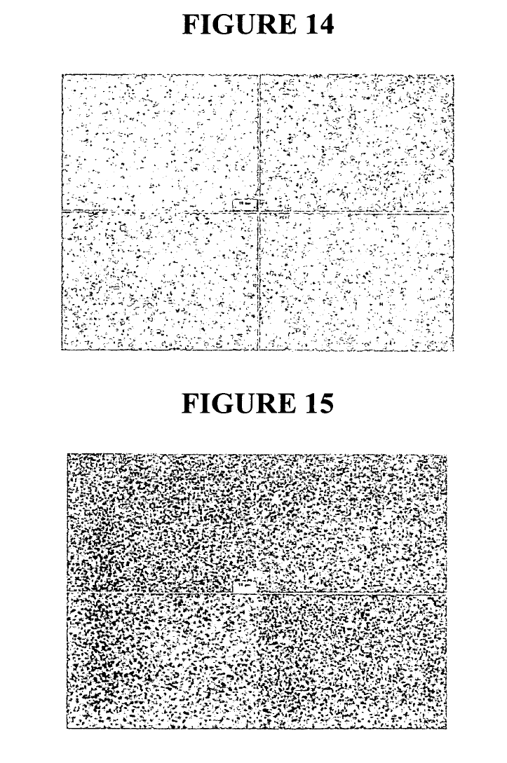

An aqueous dispersion of 10% (w/w) tacrolimus (Camida LLC) and 2% (w/w) Pluronic® F108 was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 minutes. A light micrograph using phase optics at 100× of the milled tacrolimus is shown in FIG. 14.

Following milling, the particle size of the milled tacrolimus particles was measured, in deionized distilled water, using a Horiba LA 910 particle size analyzer. The initial mean milled tacrolimus particle size was 237 nm, with a D50 of 212 nm and a D90 of 355 nm, as shown in Table 6, below. In a second measurement in distilled water following 1 week of refrigeration at <15° C., the mean tacrolimus particle size was 332 nm, with a D50 of 306 nm and a D90 of 467 nm. A light micrograph using phase optics at 100× of the milled tacrolimus following one week of storage under refrigeration is shown in FIG. 15.

TABLE 6

| | Mean | D50 | D90 |
|---|---|---|---|
| | Particle | Particle | Particle |
| Sample | Size (nm) | Size (nm) | Size (nm) |
| initial tacrolimus/Pluronic ® F108 | 237 | 212 | 355 |
| tacrolimus/Pluronic ® F108 sample | 332 | 306 | 467 |
| following 1 week refrigeration | | | |

The results demonstrate the successful preparation of a stable nanoparticulate tacrolimus formulation, as the mean particle size obtained was 237 nm, and minimal particle size growth was observed following storage.

### Example 9

The purpose of this example was to prepare a nanoparticulate tacrolimus formulation suitable for use as an injectable dosage form.

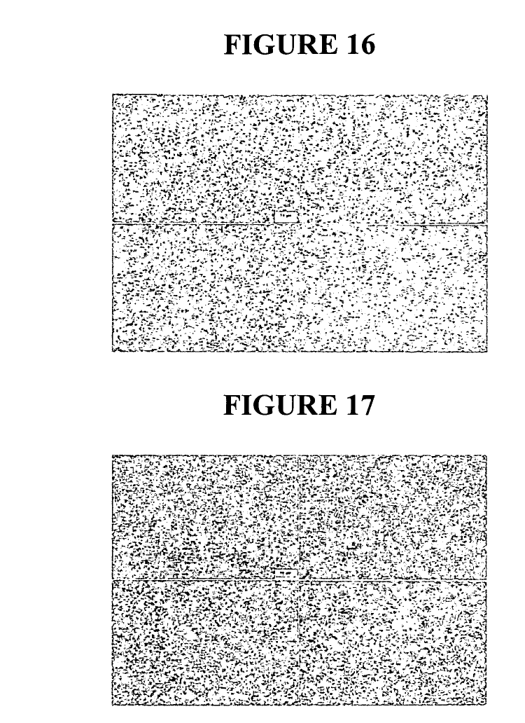

An aqueous dispersion of 10% (w/w) tacrolimus (Camida LLC) and 2% (w/w) Tween® 80 was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 minutes. A light micrograph using phase optics at 100× of the milled tacrolimus is shown in FIG. 16.

Following milling, the particle size of the milled tacrolimus particles was measured, in deionized distilled water, using a Horiba LA 910 particle size analyzer. The initial mean milled tacrolimus particle size was 208 nm, with a D50 of 191 nm and a D90 of 298 nm, as shown in Table 7, below. In a second measurement in distilled water following 1 week of refrigeration at <15° C., the mean tacrolimus particle size was 406 nm, with a D50 of 348 nm and a D90 of 658 nm. A light micrograph using phase optics at 100× of the milled tacrolimus following one week of storage under refrigeration is shown in FIG. 17.

TABLE 7

| | Mean | D50 | D90 |
|---|---|---|---|
| | Particle | Particle | Particle |
| Sample | Size (nm) | Size (nm) | Size (nm) |
| initial tacrolimus/Tween ® 80 | 208 | 191 | 298 |
| tacrolimus/Tween ® 80 sample | 406 | 348 | 658 |
| following 1 week refrigeration | | | |

The results demonstrate that this formulation is probably not preferred, as the tacrolimus particle size almost doubled after one week of storage. However, the particle size is still within the preferred size of less than 2 microns.

### Example 10

The purpose of this example is to describe injectable dosage forms comprising nanoparticulate tacrolimus and sirolimus.

An injectable composition comprising nanoparticulate tacrolimus and nanoparticulate sirolimus can be prepared by combining any of the nanoparticulate tacrolimus formulations described in Examples 1-5 or 7-9 with a nanoparticulate sirolimus composition. A nanoparticulate sirolimus composition can be made as described in US 20030054042, for “Stabilization of chemical compounds using nanoparticulate formulations.”

It will be apparent to those skilled in the art that various modifications and variations can be made in the methods and compositions of the present invention without departing from the spirit or scope of the invention. Thus, it is intended that the present invention cover the modifications and variations of this invention, provided they come within the scope of the appended claims and their equivalents.

What is claimed is:

1. An injectable nanoparticulate formulation comprising:
(a) particles of tacrolimus having an effective average particle size of less than about 2000 nm; and
(b) at least one surface stabilizer.

2. The composition of claim 1, further comprising particles of sirolimus having an effective average particle size of less than about 2000 nm and a surface stabilizer, wherein the sirolimus surface stabilizer can be the same as or different from the tacrolimus surface stabilizer.

3. The composition of claim 1, wherein the tacrolimus is selected from the group consisting of a crystalline phase, an amorphous phase, a semi-crystalline phase, a semi-amorphous phase, and mixtures thereof.

4. The composition of claim 1, wherein the effective average particle size of the nanoparticulate tacrolimus particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, and less than about 50 nm.

5. The composition of claim 1, when injected into a patient, forms a subcutaneous or intramuscular depot for long term immunosuppressant release.

6. The composition of claim 1, wherein the composition further comprises one or more pharmaceutically acceptable excipients, carriers, or a combination thereof.

7. The composition of claim 1, wherein the at least one surface stabilizer is present in an amount selected from the group consisting of from about 0.5% to about 99.999% by weight, from about 5.0% to about 99.9% by weight, and from about 10% to about 99.5% by weight, based on the total combined dry weight of the tacrolimus and at least one surface stabilizer, not including other excipients.

8. The composition of claim 1, wherein the tacrolimus is present in an amount selected from the group consisting of from about 99.5% to about 0.001%, from about 95% to about 0.1%, and from about 90% to about 0.5%, by weight, based on the total combined weight of the tacrolimus and at least one surface stabilizer, not including other excipients.

9. The composition of claim 1, comprising at least two surface stabilizers.

10. The composition of claim 1, wherein the surface stabilizer is selected from the group consisting of an anionic surface stabilizer, a cationic surface stabilizer, a zwitterionic surface stabilizer, a non-ionic surface stabilizer, and an ionic surface stabilizer.

11. The composition of claim 1, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅ dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

12. The composition of claim 1, comprising as a surface stabilizer a povidone polymer having a molecular weight of about 40,000 daltons or less.

13. The composition of claim 1, additionally comprising one or more non-tacrolimus or non-sirolimus active agents.

14. The composition of claim 1, wherein the composition redisperses in a biorelevant media such that the tacrolimus particles have an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, and less than about 50 nm.

15. The composition of claim 14, wherein the biorelevant media is selected from the group consisting of water, aqueous electrolyte solutions, aqueous solutions of a salt, aqueous solutions of an acid, aqueous solutions of a base, and combinations thereof.

16. The composition of claim 1, wherein the Tₘₐₓ of the tacrolimus, when assayed in the plasma of a mammalian subject following administration, is less than the Tₘₐₓ for non-nanoparticulate tacrolimus, administered at the same dosage.

17. The composition of claim 16, wherein:
(a) the Tₘₐₓ is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tₘₐₓ exhibited by a non-nanoparticulate tacrolimus formulation, administered at the same dosage;
(b) the composition exhibits a Tₘₐₓ selected from the group consisting of less than about 6 hours, less than about 5 hours, less than about 4 hours, less than about 3 hours, less than about 2 hours, less than about 1 hour, and less than about 30 minutes after administration to fasting subjects; or
(c) a combination of (a) and (b).

18. The composition of claim 1, wherein the Cₘₐₓ of the tacrolimus, when assayed in the plasma of a mammalian subject following administration, is greater than the Cₘₐₓ for a non-nanoparticulate tacrolimus formulation, administered at the same dosage.

19. The composition of claim 18, wherein the Cₘₐₓ is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by a non-nanoparticulate tacrolimus formulation, administered at the same dosage.

20. The composition of claim 1, wherein the AUC of tacrolimus, when assayed in the plasma of a mammalian subject following administration, is greater than the AUC for a non-nanoparticulate tacrolimus formulation, administered at the same dosage.

21. The composition of claim 20, wherein the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate formulation of the immunosuppressant, administered at the same dosage.

22. The composition of claim 1 which does not produce significantly different absorption levels when administered under fed as compared to fasting conditions.

23. The composition of claim 22, wherein the difference in absorption of the tacrolimus composition, when administered in the fed versus the fasted state, is selected from the group consisting of less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, and less than about 3%.

24. The composition of claim 1, wherein administration of the composition to a human in a fasted state is bioequivalent to administration of the composition to a subject in a fed state.

25. A method of making an injectable nanoparticulate tacrolimus composition comprising contacting tacrolimus particles with at least one surface stabilizer for a time and under conditions sufficient to provide tacrolimus particles having an effective average particle size of less than about 2000 nm.

26. The method of claim 25, wherein the contacting comprises grinding, wet grinding, homogenizing, precipitation, or supercritical fluid particle generation techniques.

27. The method of claim 25, further comprising adding a nanoparticulate sirolimus composition to the nanoparticulate tacrolimus composition, wherein the nanoparticulate sirolimus composition comprises sirolimus particles having an effective average particle size of less than about 2000 nm and a surface stabilizer, wherein the sirolimus surface stabilizer can be the same as or different from the tacrolimus surface stabilizer.

28. A method for the prophylactic treatment of organ rejection or treatment of psoriasis or other immune diseases comprising administering to a subject in need an effective amount of an injectable tacrolimus composition comprising:
(a) tacrolimus particles having an effective average particle size of less than about 2000 nm; and
(b) at least one surface stabilizer.

29. The method of claim 28, further comprising administering a nanoparticulate sirolimus composition to the subject in need, wherein the nanoparticulate sirolimus composition comprises sirolimus particles having an effective average particle size of less than about 2000 nm and a surface stabilizer, wherein the sirolimus surface stabilizer can be the same as or different from the tacrolimus surface stabilizer.

30. The method of claim 28, wherein the subject is a human.

31. A method of treating a mammal comprising administering to the mammal an effective amount of an injectable pharmaceutical composition to form a subcutaneous or intramuscular depot for long term release, wherein the composition comprises:
(a) tacrolimus particles having an effective average particle size of less than about 2000 nm;
(b) at least one surface stabilizer; and
(c) a pharmaceutically acceptable carrier.

32. The method of claim 31, further comprising administering a nanoparticulate sirolimus composition to the mammal, wherein the nanoparticulate sirolimus composition comprises sirolimus particles having an effective average particle size of less than about 2000 nm and a surface stabilizer, wherein the sirolimus surface stabilizer can be the same as or different from the tacrolimus surface stabilizer.
